(19) United States
(12) Patent Application Publication
(10) Pub. No.: US 2020/0146884 A1
(43) Pub. Date: May 14, 2020

(54) NON-SHIVERING CRYOTHERMOGENESIS METHOD FOR REDUCING ADIPOSE TISSUES

(71) Applicant: Suvaddhana Sarin LOAP, Paris (FR)

(72) Inventor: Suvaddhana Sarin LOAP, Paris (FR)

(21) Appl. No.: 16/473,569

(22) PCT Filed: Dec. 14, 2017
(86) PCT No.: PCT/EP2017/082823

§ 371 Date: Jun. 25, 2019

(30) Foreign Application Priority Data
Dec. 27, 2016 (EP) 16306821.6

(51) Int. Cl. A61F 7/10

(52) U.S. Cl. CPC A61F 7/10; A61F 2007/0056

(57) ABSTRACT

The present disclosure refers to a non-shivering cryothermogenesis method for locally reducing adipose tissue of a subject's body, including: (a) applying at least one applicator to the subject's skin; (b) applying at least one additional applicator to a local region wherein adipose tissue reduction is desired; (c) the applicator maintaining skin superficial temperature between 4° C. and 12° C.; and (d) applying the applicators for a total period of time between 20 and 60 min.

### CROSS-REFERENCE TO RELATED APPLICATIONS

This application is a National Phase Entry of International Patent Application No. PCT/EP2017/082823, filed on Dec. 14, 2017, which claims priority to European Patent Application No. 16306821.6, filed on Dec. 27, 2016, both of which are incorporated by reference herein.

### BACKGROUND AND SUMMARY

The present invention relates to a non-invasive cosmetic method activating non-shivering thermogenesis to reduce adipose tissue in a subject expecting slimming subject by controlled cooling.

Excess body fat and localized adiposity represent important social problems and many subjects affected by it seek plastic surgery to improve their appearance. However, plastic surgery is an invasive medical procedure with associated risks, which generally may allow a rapid recovery and may affect the social life. Indeed, even with regular physical exercises, it is difficult to eliminate fat from lipid-rich cells or adipose tissue, as they usually only burn their fats in extreme conditions, such as diet. However, diets bring only short-term benefits and present the disadvantage to be long, difficult, requiring to change the entire lifestyle and, lead in many cases to regain lost weight. Accordingly, various cosmetic procedures and devices have been developed to remove or reduce unwanted local subcutaneous fat.

Attention has focused on alternative methods for adipose tissue (AT) disruption including radiofrequency, ultrasound and laser treatment. In recent years, body cooling or “cryolipolysis” has been developed, it involves either environmental cold exposure (Bartelt et al.) or devices to reduce skin surface temperature to −7° C. (e.g., U.S. Pat. Nos. 9,308,120 and 7,367,341, Example 1), leading to systemic or local exposure of fatty tissues to active cooling. Cryolipolysis development was based on studies showing that intracellular lipid content varies among cell types, with dermal and epidermal cells being relatively low in unsaturated fatty acids compared to the underlying adipocytes that form the subcutaneous fatty tissue. As a result, the different cell types, e.g. lipid-rich and non-lipid-rich cells, exhibit varying degrees of susceptibility to the cold, with non-lipid-rich cells withstanding colder temperatures than lipid-rich cells. Therefore, cryolipolysis is using these properties by applying cooling means to crystallize cytoplasmic lipids, disrupt cellular integrity and induce cell death via apoptosis/necrosis, to selectively reduce AT over a period of weeks to months (U.S. Pat. No. 9,308,120).

Accordingly, due to the adipocyte cell death, methods disrupting cellular integrity as cryolipolysis present the inconveniences of:
localized bruising, frostbite, swelling or dulling of sensation may occur (which clears within a few weeks);
developing hard lump due to the crystallization of cytoplasmic lipids, requiring massaging the lump after treatment;
risks of paradoxical adipose hyperplasia (Singh et al. (2015), and Jalian et al. (2014)), which can require surgery in some severe cases;
generating fat dead cells debris, fat (lipotoxicity), cytokines and other inflammatory mediators that the organism has to gradually digest and eliminate in the weeks or months after the procedure, which restrain the treatment to a limited number of local regions at the same time.

These inconveniences lead to widely spaced treatment (e.g., several days or weeks apart) to allow the subject to eliminate said cell debris and fat, to resolve inflammation and other side effects. However, lipid-rich cells or adipose tissue are very useful as they are involved in numerous metabolism processes, such as storing excess energy as lipid, regulating systemic energy balance through the release of adipokines that target peripheral tissues (Dempersmier et al., 2015), targeting the brain to modulate appetite in response to excess energy supply (Sonna et al., 1985), and protecting tissues including muscle and liver from lipotoxicity (Haemmerle et al., 2006).

Accordingly, convenient and efficient cosmetic methods to improve subject appearance by reducing adipose tissue without causing injury to lipid rich cells (e.g. adipose tissue) were heretofore unknown. In this context, the applicant surprisingly found a method using controlled-cooling to boost energy expenditure via fat metabolism and non shivering thermogenesis, which does not present the inconvenient related to lipid rich cell disruption described above. Accordingly, the method of the invention was shown to promote reduction of adipose tissue without lipid rich cell disruption, reducing waist circumference with a single session of multiple applications, or to cumulate reduction of waist circumference with repeated sessions (even daily repeated sessions) with aesthetic purpose. Others advantages of the present method over prior art methods, is that it does not promote inflammatory reaction or loose skin, and it increases venous return and it is active on lipomastia.

The present invention relates to a non-shivering cryothermogenesis method for locally reducing adipose tissue of a subject's body, comprising:
(a) applying at least one applicator of a cooling mean to subject's skin to regions wherein subcutaneous adipose tissue is rich in brown adipocytes (i.e. Brown Adipose Tissue or BAT) and/or in white adipose tissue (WAT) containing brown adipocytes; and
(b) applying further applicators of said cooling mean to local region wherein adipose tissue reduction is desired; and
(c) said applicators of said cooling mean maintaining skin superficial temperature between 4° C. and 12° C., preferentially between 6° C. and 10° C., more preferentially about 8° C.; and
(d) applying said applicators for a total period of time comprised between 20 and 60 minutes, preferentially between 30 and 50 minutes, more preferentially about 40 minutes.
   Advantageously, the method according to the invention further comprise in said step b) applying additional applicators of said cooling mean along the large vascular axes of the circulatory system, such as femoral artery, tibial artery, subclavian artery, axillary artery, brachial artery . . . en prenant soin de ne pas les comprimer

The term “cryothermogenesis” refers herein to a biological process activated by cold which induce or enhance thermogenesis, without causing cell death. By “thermogenesis” it is meant a process occurring in all warm-blooded animals, and consisting in cell producing heat from fatty acids and carbohydrates. During these metabolic process non-toxic end-products are released (e.g. H₂O and CO₂) and no inflammatory reaction is induced.

The terms “non-shivering cryothermogenesis” are defined as a cold-induced increase in heat production not associated with the muscle activity of shivering. Accordingly, as shown herein no significant difference in the level of inflammatory biomarker(s) can be observed before, during and after the application of the method according to the invention.

By “reducing adipose tissue” it is meant herein that adipose tissue volume of at least one body part of a subject is reduced. This reduction in adipose tissue can be determined by measuring the thickness of the skin with an adipometer or by measuring circumference of the treated body part of the subject.

The term “inflammatory biomarker(s)” as used herein relates to biomarker(s) indicative of the presence of an inflammatory state, e.g. neopterin, sedimentation rate, C-reactive protein (CRP), and additional cell markers and cytokines identifiable by a skilled person.

The term “subcutaneous adipose tissue” means tissue lying underneath the dermis and includes adipocytes (fat cells) and subcutaneous fat. By “subcutaneous adipocytes tissue rich in brown adipocytes” refers to areas localized along the spine (cervical, paravertebral), supraclavicular, axillary, pericardial, pericardial/renal/adrenal, trachea-esophageal, mediastinal, intercostal and mesenteric (FIG. 1). The expression “white adipose tissue (WAT) containing brown adipocytes” or “brite adipocytes” (i.e. brown in white) or beige adipocytes refer to all areas of white adipose tissue that contains brown adipocytes. Beigeing or browning adipocytes refers to white adipocytes transformation into brown adipocytes by various factors, including cold.

The term “brown adipocyte” relates to cells present in BAT that are specialized in heat production (thermogenesis) in mammals. Two principal types of adipose tissue have been described, WAT and BAT. The most abundant adipose cell types are the white adipocytes that contain a single intracellular lipid droplet and localize to specific depots within the body. In contrast to WAT, BAT adipocytes express uncoupling proteins that permit mitochondria to metabolize fat via β-oxidation to generate heat (Avram et Harry, 2009). Uncoupling proteins 1, when activated, short circuits the electrochemical gradient that drives ATP synthesis and thereby stimulates respiratory chain activity. Heat is generated from the combustion of available substrates and is distributed to the rest of the body through the circulation. Similarly, “brown-like’ adipocytes” or “beige fat cells” where also identified in adipose tissue and refers to cells containing significant amounts of Uncoupling proteins 1, providing them thermogenic capacities http://www.nature.com/nm/journal/v19/n10/full/nm.3361.html—ref10.

By “local region wherein adipose tissue reduction is desired” it is meant a defined region or area comprising adipocytes, and where cooling means will be applied to specifically reduce the adipose tissue present in this area, or to specifically activate thermogenesis through brown adipocytes and/or WAT containing brown adipocytes. According to a preferred embodiment, said local region are selected from the stomach, love handles, inner thighs, inner knees, butt, inner arms, chin, breast area, back and neck. Therefore, the method according to the invention by reducing adipose tissue in a subject can reduce circumference of the waist, hips, upper thigh, lower thigh, calf, forearms, biceps, chest and/or shoulder, preferentially the circumference of the waist, hips and/or upper thigh.

By “maintaining skin superficial temperature” it is meant herein that the cooling mean is adapted to cool the surface of the skin to a specific temperature and to maintain it by adapting the applied temperature. However, it will be obvious for a skilled person to adapt the applied temperature according to the duration of application, and conversely, to adapt the duration of application according to the applied temperature. Said skin superficial temperature can be determined by any mean allowing to measure temperature, which are well known by one skilled in the art. For examples such mean allowing to measure temperature can be selected from infrared thermometer or laser thermometers or non-contact thermometers. Therefore, by maintaining skin superficial temperature at the temperature according to the invention, adipocyte cells are preserved from freezing-induced cell death, and any disruption of adipocyte cells is prevented.

By “about 8° C.” it is meant a skin superficial temperature of 8° C.±2° C., preferentially 8° C.±1° C. By “about 40 minutes” it is meant a period of time of 40 minutes±4 minutes, preferentially 40 minutes±2 minutes. Obviously, the skilled person performing the claimed method would be capable to adjust parameters in order to maintain the skin superficial temperature to about 8° C.

By “cooling mean” it is referred herein to any mean capable to cool and/or apply to a body part of a subject a specific set up temperature during a certain period of time, i.e. by removing heat or applying cold. The term “applicator” refers to a part of a cooling mean that can be applied directly to a subject's skin and from which generated cold air is expelled to cool the skin surface of the subject. Of course, it would be obvious to the skilled person to adapt the shape or the size of said applicator to the local area to be cooled. Preferably, said applicators are able to apply negative pressure to form a sealed chamber in contact to the skin of a subject, wherein the specific set up temperature is applied in said chamber. Also, according to local region to be treated by the method according to the invention, the said applicators would be chosen according to the surface of the skin to be covered. Such cooling devices and applicators are commercially available and are for example supplied by Beijing ADSS Development Co., Ltd, or Beijing Jontelaser Technology Co., Ltd, or Beijing Himalaya Medical Technology Co., Ltd.

In a particular embodiment, to perform the method according to the invention, applicators of said cooling mean are applied to said subject's skin in a number comprised at least 2, preferentially between 5 to 14, more preferentially between 8 to 12. Preferentially, said applicator of said cooling mean cover a surface area comprised between 60 and 90 cm², preferentially between 70 and 80 cm². Advantageously said applicator has a rectangular shape. It will be obvious for a skilled person to adapt the shape and/or size of said applicators to said local region wherein adipose tissue reduction is desired.

Moreover, it will be obvious for a skilled person to adapt the number and/or the shape and/or the size of applicators to the subject's build in order to cover the largest surface area of the subject's skin. In a preferred embodiment, said applicators of said cooling mean cover between 30% and 70%, preferentially between 40% and 55% of the back or the front of the subject upper body (i.e. from the neck to the lower back or pelvis). In a preferred embodiment, said applicators of said cooling mean cover between 60 cm² and 120 cm², preferentially between 70 cm² and 100 cm² of the subject body. In a preferred embodiment, said applicators cover between 19% and 33.5%, preferentially between 22.5% and 30% of the back of the subject. In a preferred embodiment, said applicators cover between 14% and 31.5%, preferentially between 17.5% and 25% of the front of the subject.

In a preferred embodiment, the temperature applied by said applicator in the method according to the invention is comprised between −15° C. to 5° C., preferentially −10° C. to 0° C., more preferentially −9° C. to −3° C. In a particular embodiment, time periods of step d) of the method according to the invention is divided in two or three subperiods, preferentially three subperiods, said subperiods during between 8 and 25 minutes each, preferentially during 10 or 20 minutes each. In a preferred embodiment, the period of time of step d) of the method according to the invention is comprised between 25 and 75 minutes, between 30 and 65 minutes, preferentially between 35 and 55 minutes, more preferentially about 40 minutes.

In a preferred embodiment, the method according to the invention comprises three subperiods consisting in:

i. about 10 minutes at about −10° C.; and

ii. about 10 minutes at about −8° C.; and

iii. about 20 minutes at about −7° C.

In another embodiment, said applicators of said cooling mean according to the invention are applied to the subject's skin with a suction pressure comprised between 20 and 300 kPa, preferentially between 100 kPa and 200 kPa. Preferentially, said pressure is comprised between 20 kPa and 100 kPa when the thickness of the skin thickness of a subject is inferior or equal to about 3 cm, and is comprised between 101 kPa and 300 kPa when the thickness of the skin of a subject is superior to about 3 cm. The term “about 3 cm” meant that the thickness of the skin is 3 cm±0.3, preferentially 3 cm±0.2. Preferentially, said pressure should vary between 20 kPa to 100 kPa when said applicators of said cooling mean are applied along the large vascular axes of the circulatory system, such as femoral artery, tibial artery, subclavian artery, axillary artery, brachial artery . . . .

The expression “the thickness of the skin” refers herein to the measurement of the thickness of the skin by an adipometer. It is known from the skilled person that adipometer measurements are used as a guide for calculating the amount of subcutaneous fat. However, it would be obvious for a skilled person to adapt threshold values with measurements obtained by any other means.

In a preferred embodiment, when said applicators are of rectangular shape, the method according to the invention comprised an additional step following said steps a) and/or b), wherein said applicators of rectangular shape are moved by 90 degrees and applied for an additional period of time. Advantageously, said additional step following said steps a) and/or b) is applied when the thickness of the skin of said subject is superior to about 3 cm.

Another object of the present invention is a cryothermogenesis method for reducing adipose tissue of a subject's body, comprising the step of:
(a) firstly applying the method according to the invention to the back of the subject; and then
(b) secondly applying the method according to the invention to the front of the subject.

Another object of the present invention is a cryothermogenesis method for locally reducing adipose tissue of subject's body, comprising repeating the method according to the invention to the subject's skin a plurality of times until the desired reduction in adipose tissue at a local region has been achieved, wherein said repeating application can be applied as soon as said adipose tissue recover their temperature of prior said step (a) or (b). Preferentially, said plurality of times are spaced of about 60 minutes time intervals, or 1 hour time intervals, 12 hour time intervals, or 1 day time intervals, preferentially 1 day time intervals.

### BRIEF DESCRIPTION OF THE DRAWINGS

FIG. 1. Percentage change in (A) waist circumference, and (B) thigh circumference, following single abdominal applications of tissue cryotherapy (n=17; treatment duration ca 1.33 h; time following treatment, ≤1 h). Error bars indicate SD.

FIG. 2. Individual responses to tissue cryotherapy as a function of age in subjects receiving single applications of tissue cryotherapy (trendline added, P=0.2).

FIG. 3. Decrement in (A) waist circumference, (B) weight, and (C) BMI following three sequential daily tissue cryotherapy treatments (n=12). Error bars indicate SD.

FIG. 4. Progressive decline in waist circumference (expressed as %) in five subjects undergoing repeat tissue cryotherapy treatments over four (subject 1, M, 59 years of age), five (subjects 2, 3, 4; F, 47 years; F, 71 years; F, 52 years), or seven (subject 5, M, 51 years) days. The mean decline after 5 treatments was 6.1%.

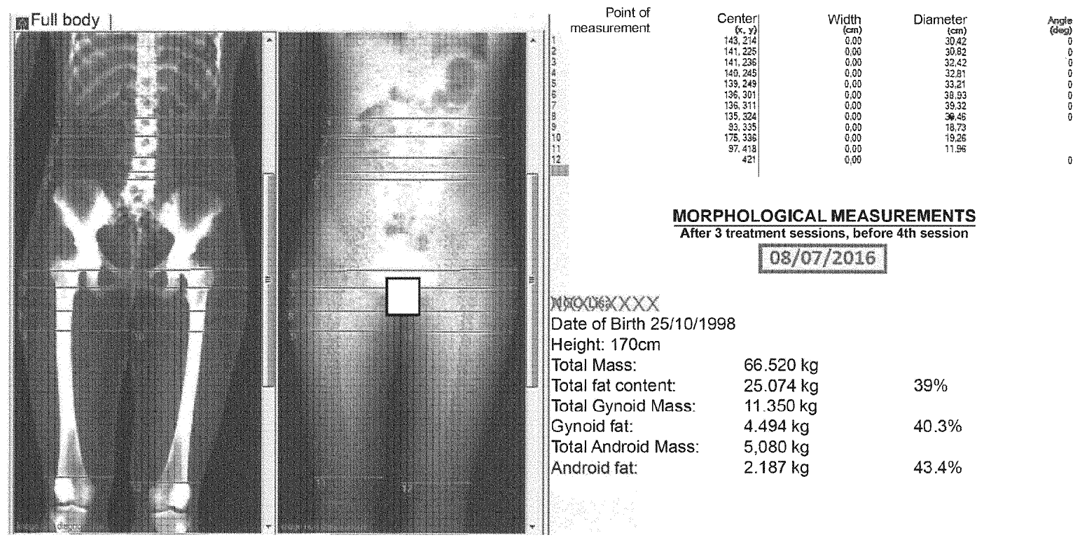

### DETAILED DESCRIPTION AND EXAMPLES

### Example 1

### Effect of the Cryothermogenesis Procedure on Waist Circumference

Methods

1. Subjects and Ethical Permissions

In this retrospective study data for 28 participants (9 M/19 F), mean age 51.5 (range 33-71 years) were analyzed. All had a BMI consistent with overweight. Thermogenesis method, a non-invasive procedure, does not require formal institutional ethical approval.

2. Tissue Cryotherapy Procedure

Treatment employed a commercial tissue cryotherapy device (FG6601-006, ADSS, Republic of China) equipped with multiple cooling probes (cooling area 20×8 cm). Weight, height, and waist and thigh (left) circumferences were recorded; BMI was calculated automatically using a commercial apparatus (model SC240MA, Tanita, Japan). A layer of wetted paper (glycerol-based wetting membrane, ETG-111-200, Freezefats, Republic of China) was applied to the lower back of the reclining subject, followed by symmetrical application of four to six pairwise probes accompanied by gentle suction to improve contact. Cooling temperature was set to −10° C., declining to −7° C. over 20 minutes; application duration was 40 minutes. Subjects then lay supine and the treatment was repeated on the lower abdomen, again for 40 minutes. Oral temperature, a proxy for core temperature, was recorded before and after the procedure. Local skin surface temperatures were recorded immediately after removal of the cooling probe and measurement showed that it was maintained around +8° C. and did not fall below +4° C. Weight, waist and thigh circumference, body weight, and BMI were recorded again after the procedure. 12 subjects volunteered for triple daily applications. Five participants (3 F and 2 M; aged 47, 51, 71; and 51, 57 years, respectively) volunteered for more extended daily applications, and the procedure was repeated on a daily basis for a total of four, five, or seven applications, with systematic recording as before.

3. Statistics

Statistical analyses employed normalized means and SD. Results were evaluated using the Student t test; statistical significance was set at P≤0.05.

4. Local Cooling Produces Decrements in Waist Circumference

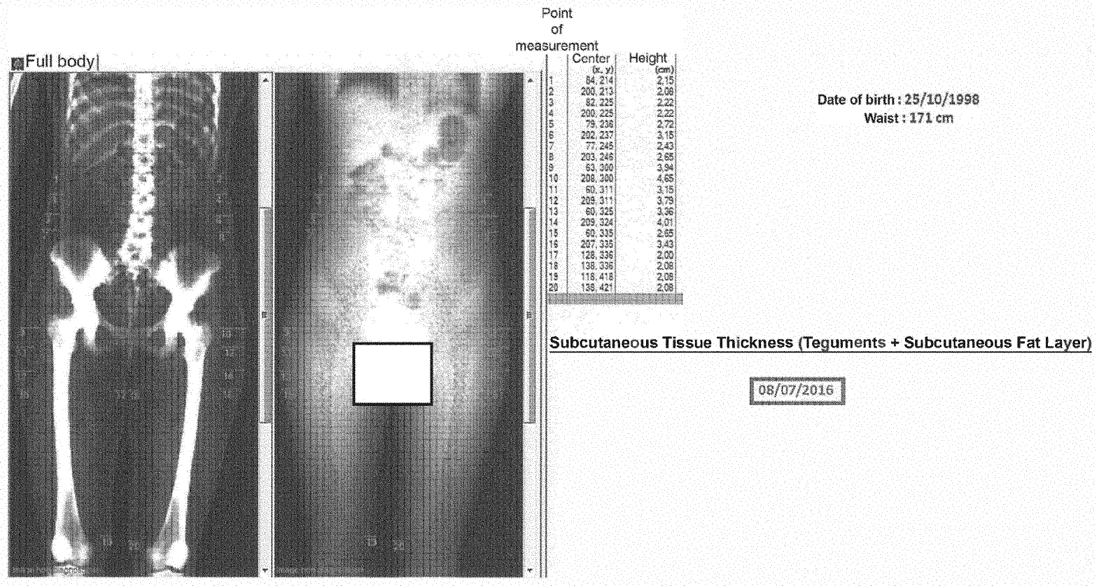

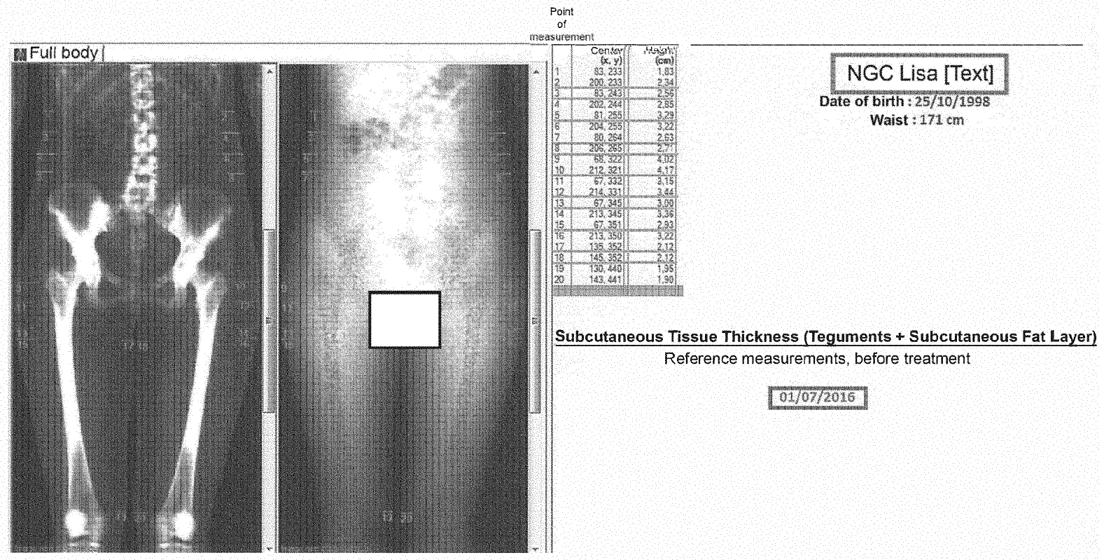

Waist circumference was recorded before and after single applications of local abdominal tissue cooling (cryothermogenesis method) in a series of 17 subjects. As shown in FIG. 1A, there was a mean reduction of 3.0% of waist circumference following the procedure. The decline was of high statistical significance (P=<0.0001). By contrast, there was no change in thigh circumference (FIG. 1B). Core body temperature (oral) rose from mean 35.8 to mean 36.1 (+0.3° C.), but the change was not significant. Changes in weight and BMI with single applications fell short of statistical significance (not presented; see below).
As shown in FIG. 2, there was a trend towards a reduction in efficacy as a function of age. However, the trend was not of statistical significance (P=˜0.2): some older individuals displayed substantial reductions in waist circumference and some young subjects displayed low responses. There were no significant gender differences.

5. Multiple Applications Produce Loss of Waist Circumference

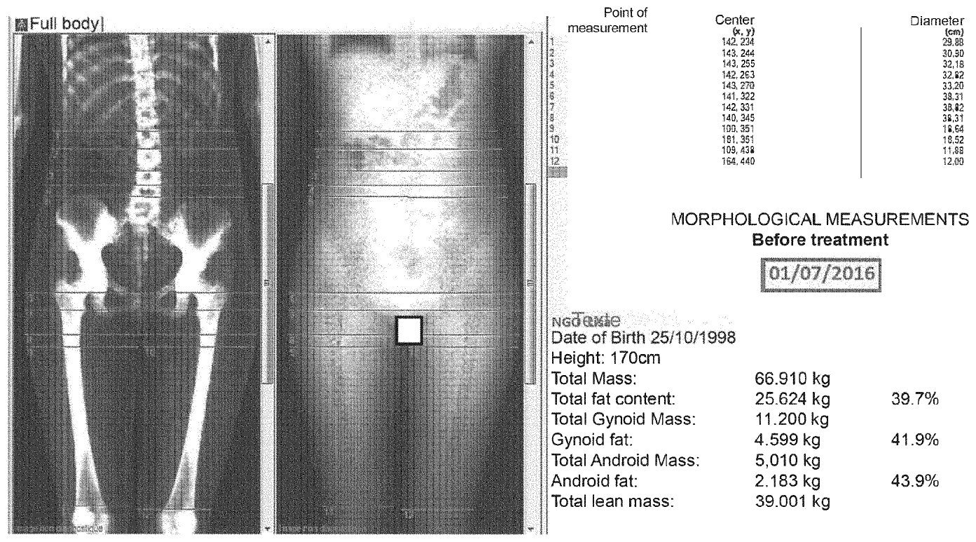

Twelve subjects undertook subsequent treatments on days 2 and 3 under the same conditions and for the same duration as on day 1. As shown in FIG. 3, in addition to a significant decline in waist circumference (P=0.0068), highly significant reductions in both body weight (P=<0.001) and BMI (P=0.013) were observed. The extent of mean reduction in waist circumference, 4.2%, exceeded the mean reduction obtained by single applications (3.0%, P=NS versus single application).
To address a possible delay-/time-dependent effect, nine subjects receiving a single treatment underwent further measurements 5 to 10 days later but without further tissue cryotherapy. Given the small number of subjects, the extent of the reduction in waist circumference (3.9%, SD=3.09) did not achieve statistical significance versus the reduction observed immediately following single treatment (3.0%, SD=2.71), although there was trend towards an increase.
Five subjects (3 F, 2 M) volunteered for repeat daily treatments over periods of 4, 5, and 7 days. As shown in FIG. 3, repeat treatment was accompanied, in all five subjects, by a progressive loss of waist circumference (mean loss of 6.1% immediately following five treatments), to be compared against a 3% mean loss immediately following a single treatment. The difference in waist loss following four to seven treatments was significantly greater than from single treatments (P=<0.01).

6. Biomedical Parameters

Local skin temperatures immediately beneath the cooling probe were variable within the region, but did not fall below +6° C. There was some local reddening of the skin consistent with increased local blood circulation. No superficial skin damage of any type was observed. Comprehensive blood analysis was carried out on four subjects before and 3 days after treatment. Profiles before and after treatment were unremarkable. Importantly, except as noted, there was no indication of inflammation, macrophage activation, or abnormal lipid profiles. Therefore the cryothermogenesis method is not associated with systemic inflammation or changes in blood lipid profiles. In addition, no participant reported any adverse events.

7. Conclusion

Single applications of local abdominal tissue cooling (cryothermogenesis method) were found to produce a significant tissue reduction. All subjects responded to single treatments by a reduction in waist circumference (mean ˜3%) that was statistically significant (P=<0.0001). There was no evident M/F gender difference in response. There was an apparent trend towards to reduced efficacy with age; however, the trend was not statistically significant, and participants among the oldest in the group displayed responses to single treatments.

Results show further progressive loss of AT with multiple applications. A larger reduction in circumference was observed following three treatments (˜4.2%), and this was accompanied by decrements in both body weight (˜0.75%) and BMI (˜0.8%) that were statistically significant. The reduction in BMI indicates selective loss of AT volume. The reduction in waist circumference after three treatments (˜0.4%) was consistent with the decline in total body weight (˜0.75%).

In five subjects receiving extended rounds of treatment (four to seven), there was evidence for progressive decrements in waist circumference (˜6% after five treatments), arguing that multiple treatments at daily intervals produce cumulative benefits. No consistent changes in biochemical parameters or adverse events were reported for the participants in this study. These results confirm that local cooling of abdominal tissue produces selective loss of AT volume.

In contrast to earlier reports that there is no evident change in body fat immediately after treatment, the results showed significant beneficial effects within 1 h of treatment, a time-course too rapid to be explained by cell death and clearance. In addition, multiple daily treatments produced progressive losses of waist circumference, bodyweight, and BMI which is incompatible with the cell disruption. Furthermore, biochemical analysis revealed no evidence of markers of inflammation or macrophage activation in treated subjects, showing adipocyte cell volume reduction via thermogenesis, with conservation of cellular integrity. In support, complete maintenance of core body temperature (+0.3° C.) was recorded despite intense local cooling, attesting that thermogenesis is taking place. Results therefore point to energy expenditure via cold-induced fat metabolism and thermogenesis, with consequent reduction in adipocyte cell volume and AT mass.

Regarding the safety of the method according to the invention, no consistent changes in biochemical parameters were observed following treatment, including markers of inflammation. No adverse effects were reported by the participants.

### Example 2

### Inflammatory Biomarkers Measurements

Method

1. Biomedical Parameters

Comprehensive blood analysis was carried out on subjects before and 3 days after the daily treatment applied daily during 5 to 10 days. All profiles before and after treatment (±4 days) were unremarkable. Importantly, there was no indication of inflammation or macrophage activation.

Results

TABLE 1
Measure of Neopterine before and
after cryothermogenesis treatment

| | | Neopterine (nmol/l) |
|---|---|---|
| Subject 1 | before | 3.3 |
| | after | 8 |
| Subject 2 | before | 2.9 |
| | after | 5.2 |
| Subject 3 | before | 9 |
| | after | 6.5 |
| Subject 4 | before | 4 |
| | after | 6.6 |
| Subject 5 | before | 8.1 |
| | after | 7.4 |

TABLE 2
Measure of the sedimentation rate (first hour
and second hour) and C-reactive protein (CRP)
before and after cryothermogenesis treatment

| | | Sedimentation rate | CRP |
|---|---|---|---|
| Subject a | before | 7 and 19 | <5 |
| | after | 6 and 13 | <1 |
| Subject b | before | 4 and 9 | 3 |
| | after | 9 and 26 | 5 |
| Subject c | before | 3 and 6 | <5 |
| | after | 3 and 6 | <5 |
| Subject d | before | 6 and 14 | <5 |
| | after | 6 and 15 | 2 |
| Subject e | before | 2 and 6 | 5 |
| | after | 3 and 5 | <1 |

Conclusions

Results of table 1 and 2 showed that cryothermogenesis method is not associated with systemic inflammation as neopterine (normal value: <25 nmol/I, the sedimentation rates (normal values: first hour 1 to 20 and second hour 4 to 40) and CRP (normal value: <6 mg/L), which are well known by the skilled person, are in the range of their respective normal values. In addition, no participant reported any adverse events.

### REFERENCES

M. M. Avram, R. S. Harry, Cryolipolysis for subcutaneous fat layer reduction. Lasers Surg. Med. 41 (2009) 703-708.
A. Bartelt, O. T. Bruns, R. Reimer, H. Hohenberg, H. Ittrich, K. Peldschus, M. G. Kaul, U. I. Tromsdorf, H. Weller, C. Waurisch, A. Eychmuller, P. L. Gordts, F. Rinninger, K. Bruegelmann, B. Freund, P. Nielsen, M. Merkel, J. Heeren, Brown adipose tissue activity controls triglyceride clearance. Nat. Med. 17 (2011) 200-205.
J. Dempersmier, A. Sambeat, O. Gulyaeva, S. M. Paul, C. S. Hudak, H. F. Raposo, H. Y. Kwan, C. Kang, R. H. Wong, H. S. Sul, Cold-inducible Zfp516 activates UCP1 transcription to promote browning of white fat and development of brown fat. Mol. Cell. 57 (2015) 235-246.
G. Haemmerle, A. Lass, R. Zimmermann, G. Gorkiewicz, C. Meyer, J. Rozman, G. Heldmaier, R. Maier, C. Theussl, S. Eder, D. Kratky, E. F. Wagner, M. Klingenspor, G. Hoefler, R. Zechner, Defective lipolysis and altered energy metabolism in mice lacking adipose triglyceride lipase. Science. 312 (2006) 734-737.
H R Jalian; M M Avram; L Garibyan; M C Mihm; R. R Anderson. Paradoxical Adipose Hyperplasia After Cryolipolysis. JAMA Dermatol. 2014; 150(3):317-319.
S M Singh, E R Geddes, S G Boutrous, R D Galiano, P M Friedman. Paradoxical adipose hyperplasia secondary to cryolipolysis: An underreported entity? Lasers Surg Med. (2015); 47(6):476-8.
L. A. Sonna, J. Fujita, S. L. Gaffin, C. M. Lilly, Invited review: Effects of heat and cold stress on mammalian gene expression. J. Appl. Physiol. (1985.) 92 (2002) 1725-1742.

1. A non-shivering cryothermogenesis method for locally reducing adipose tissue of a subject's body, comprising:
(a) applying at least one cooling applicator to the subject's skin to region wherein subcutaneous adipocytes tissue is rich in brown adipocytes and/or in white adipose tissue (WAT) containing brown adipocytes;
(b) applying at least one additional cooling applicator to a local region wherein adipose tissue reduction is desired, if said local region differs from step (a);
(c) said cooling applicator maintaining skin superficial temperature between 4° C. and 12° C.; and
(d) applying said cooling applicators for a total period of time comprised between 20 and 60 min.

2. The method of claim 1, wherein said cooling applicator applies a temperature comprised between −15° C. to 5° C.

3. The method of claim 1, wherein said time period of step d) is divided in two or three subperiods, each of said subperiods being between 10 and 20 minutes.

4. The method of claim 1, wherein said three subperiods consist in:
i. applying a temperature of −10° C. during 10 minutes; and
ii. applying a temperature of −8° C. during 10 minutes; and
iii. applying a temperature of −7° C. during 20 minutes.

5. The method of claim 1, wherein said local region is selected from the stomach, love handles, inner thighs, inner knees, butt, inner arms, chin, breast area, back and neck.

6. The method of claim 1, wherein 5 to 14 of said cooling applicators are applied to said subject's skin.

7. The method of claim 1, wherein said cooling applicators are applied to the subject's skin with a pressure comprised between 50 kPa and 300 kPa.

8. The method of claim 7, wherein said pressure is comprised between 50 and 150 kPa when the thickness of the skin of a subject is inferior or equal to 3 cm, and is comprised between 151 and 300 kPa when the thickness of the skin of a subject is superior to 3 cm.

9. A cryothermogenesis method for reducing adipose tissue of a subject's body, comprising the step of:
(a) firstly applying a non-shivering cryothermogenesis method to a back of the subject; and then
(b) secondly applying the non-shivering cryothermogenesis method to a front of the subject;
the non-shivering cryothermogenesis method comprising:
(i) applying at least one cooling applicator to the subject's skin to region wherein subcutaneous adipocytes tissue is rich in brown adipocytes and/or in white adipose tissue (WAT) containing brown adipocytes;
(ii) applying at least one additional cooling applicator to a local region wherein adipose tissue reduction is desired, if said local region differs from step (i);
(iii) said cooling applicator maintaining skin superficial temperature between 4° C. and 12° C.; and
(iv) applying said cooling applicators for a total period of time comprised between 20 and 60 min.

10. A non-shivering cryothermogenesis method for locally reducing adipose tissue of a subject's body, comprising:
(a) applying at least one cooling applicator to the subject's skin to region wherein subcutaneous adipocytes tissue is rich in brown adipocytes and/or in white adipose tissue (WAT) containing brown adipocytes;
(b) applying at least one additional cooling applicator to a local region wherein adipose tissue reduction is desired, if said local region differs from step (a);
(c) said cooling applicator maintaining skin superficial temperature between 4° C. and 12° C.;
(d) applying said cooling applicators for a total period of time comprised between 20 and 60 min; and
(e) repeating the method of steps (a)-(d) to the subject's skin a plurality of times until a desired reduction in the adipose tissue at said local region has been achieved, wherein said application can be repeated as soon as said adipose tissue recover their temperature of prior said step (a) or (b).

11. The method of claim 9, wherein said plurality of times are spaced of 1 hour time intervals, 12 hour time intervals or 1 day time interval.
